(19) United States
(12) Patent Application Publication
(10) Pub. No.: US 2026/0151646 A1
(43) Pub. Date: Jun. 4, 2026

(54) PORTABLE PHOTOTHERAPY SYSTEM AND METHODS OF USING THE SAME

(71) Applicant: KidTech, Inc., Houston, TX (US)

(72) Inventors: Bo HOOD, Lake Jackson, TX (US); Aidan FINCHER, Spring, TX (US); Thomas Li CHEN, Richmond, TX (US)

(21) Appl. No.: 19/460,178

(22) Filed: Jan. 26, 2026

Related U.S. Application Data
(63) Continuation of application No. PCT/US2024/039868, filed on Jul. 26, 2024.
(60) Provisional application No. 63/629,964, filed on Jul. 26, 2023.

(51) Int. Cl. A61N 5/06

(52) U.S. Cl. CPC A61N 5/0621; A61N 2005/0637; A61N 2005/0645; A61N 2005/0652; A61N 2005/0663; A61N 2005/0664

(57) ABSTRACT

The embodiments described herein relate to a phototherapy system including a garment shell configured to be worn by an infant and including a first pocket disposed on an inner surface thereof. The phototherapy system may include a light panel configured to be removably disposed in a first pocket. The garment may be configured to position and/or orient the light panel such that a first portion of the light panel aligns with a back of the infant and a second portion of the light panel aligns with a chest of the patient. The light panel may include a plurality of light sources arranged in a grid formation and coupled to a circuit board. The light panel and/or the circuit board may be disposed in a protective shell. The protective shell may include a plurality of cut outs which can reduce an overall weight of the light panel and/or impact at least one a light path or irradiance of the plurality of light sources. The phototherapy system can include a controller configured to adjust an output of the light panel in response to at least one of user input or data collected.

### CROSS-REFERENCE TO RELATED APPLICATIONS

This application is a continuation of International Application No. PCT/US2024/039868, filed Jul. 26, 2024, entitled “Portable Phototherapy System and Methods of Using the Same,” which claims the benefit of and priority to U.S. Provisional Application No. 63/629,964, filed Jul. 26, 2023, entitled “Mobile Phototherapy System for Baby Treatments”, the disclosures of which are hereby incorporated by reference in their entirety.

### TECHNICAL FIELD

Embodiments herein relate to a garment including a phototherapy system for providing phototherapy to a patient and methods of using the same.

### BACKGROUND

Phototherapy is the delivery of light to a patient and can be used to treat a variety of diseases. For example, phototherapy can be used to reduce the appearance of psoriasis and eczema symptoms. Phototherapy can be safe for all ages and therefore is also a common treatment for newborn jaundice. However, there are common issues with conventional phototherapy treatment systems for infants such as dehydration, hypothermia, requirement of eye protector, lack of parent-child bonding, interruption of feeding during treatment, and/or interruption of treatment during feeding.

### SUMMARY

In some embodiments, an apparatus for delivery light therapy to an infant may include a garment shell configured to be opened such that the infant can be disposed inside the garment shell. The garment shell includes a first pocket disposed on an inner surface of the garment shell, the first pocket defining a space in which a light panel is configured to be removably disposed. The light panel includes a plurality of light sources configured to emit light at a predetermined wavelength and intensity. The space of the first pocket extending from a back side of the garment shell to a front side of the garment shell such that when the infant is disposed inside the garment shell and the light panel is disposed in the first pocket, a first portion of the light panel is aligned with a back of the infant and a second portion of the light panel is aligned with a chest of the infant. The garment shell may further include a second pocket defining a space on the garment shell, the second pocket configured to hold a battery and a controller operatively coupled to the light panel. The garment shell may further include a mesh forming a portion of the garment shell and configured to allow at least one of light or heat emitted by the light panel to exit the garment shell through the mesh.

In some embodiments, an apparatus comprises a flexible circuit board; a plurality of light sources arranged in an ordered configuration, the plurality of light sources coupled to a first side of the flexible circuit board; and a flexible protective shell coupled to the flexible circuit board and the plurality of light sources. The protective shell disposed on a second side of each of the light sources of the plurality of light sources and the second side of the flexible circuit board, the flexible protective shell including a plurality of indentations disposed between at least some adjacent light sources of the plurality of light sources. The plurality of light sources configured to be disposed near a surface of skin of a infant and emit light on the surface of the skin of the infant. The plurality of light sources having a viewing angle and being spaced from one another such that an intensity of light emitted from the plurality of light sources is above a predetermined threshold at the surface of the skin of the infant.

In some embodiments, a system comprises: a light panel configured to be disposed in a garment for an infant, the light panel configured to emit light as part of a light therapy session to treating a disease in the infant; one or more sensors disposed in the garment; and a controller operatively coupled to the light panel, the one or more sensors, and an external device, the controller configured to: receive, from the external device, information associated with a light therapy session; send a signal to the light panel to cause the light panel to emit light based on the information associated with the light therapy session; monitor, via the one or more sensors, one or more signals including at least one of a biosignal of the infant or a signal related to an environment inside the garment; determine whether the one or more signals crosses a predetermined threshold; and adjust an output of the light panel in response to at least one of the one or more signals crossing the predetermined threshold.

### BRIEF DESCRIPTION OF THE DRAWINGS

FIG. 1 is a schematic block diagram of a system for delivering phototherapy to a patient, according to embodiments.

FIG. 2 is a schematic block diagram of a light panel for delivering light to skin of a patient, according to embodiments.

FIG. 3 is a schematic block diagram of a garment shell configured to hold a light panel for delivering phototherapy to an infant wearing the garment shell, according to embodiments.

FIG. 4 shows a garment shell worn by an infant and configured to hold a light panel to provide phototherapy to the infant, according to embodiments.

FIGS. 5A-5B show a front view and a back view, respectively, of a garment shell configured to hold a light panel for delivering phototherapy to an infant, according to embodiments. FIG. 5C is a photograph of the garment shell showing a pocket in which the light panel is configured to be removably disposed, according to embodiments.

FIGS. 6A-6B show a front side and a back side, respectively, of a garment shell configured to hold a light panel for delivering phototherapy to an infant, according to embodiments.

FIG. 7 shows a schematic diagram of a light panel including a plurality of light sources and a protective shell, according to embodiments.

FIG. 8 is an exploded view of a light panel including a plurality of light sources and a protective shell, according to embodiments.

FIGS. 9A-9B show different configurations of light panels, according to embodiments.

FIGS. 10A-10B show different configurations of light panels, according to embodiments.

FIG. 11 shows a protective shell configured to be coupled to a light panel, the protective shell including a light absorber, according to embodiments.

FIG. 12A is a diagram of a protective shell that includes cut outs disposed over the light sources, according to embodiments. FIG. 12B is a diagram of a protective shell that includes cut outs disposed between adjacent light sources, according to embodiments.

FIG. 13 is a diagram of a light panel having a shape that corresponds to a pocket of a garment shell, according to embodiments.

FIGS. 14A-14C show examples of a user interface for controlling a light panel of a phototherapy system, according to embodiments.

FIG. 15 is a flow chart diagram of method for delivering phototherapy using a phototherapy system, according to embodiments.

### DETAILED DESCRIPTION

Phototherapy (also referred to as light therapy) can be used to treat a variety of different conditions including skin diseases such as psoriasis, eczema, vitiligo, mycosis fungoides and can also be used to treat jaundice. According to American Association of Pediatrics (AAP) and several other publications, about 60% of the infants born in United States of America (USA) each year become clinically jaundiced or with hyperbilirubinemia. Jaundice or hyperbilirubinemia is the result of increased production and reduced elimination of bilirubin from the newborn. For some infants, if it is not treated, the bilirubin level continues to increase to a dangerous level. Excessive level of bilirubin leads to brain disorder which is known as Kernicterus and Bilirubin Induced Neurological Disorder (BIND) and can be fatal.

When the body is exposed to light having certain parameters (e.g., wavelength and intensity), the body can transform unconjugated bilirubin into compounds that are more easily excreted by the body through urine and feces. Effectiveness of phototherapy depends on four main factors, which are (1) irradiance or light intensity, (2) color or wavelength of the light, (3) skin coverage or exposed area, and (4) time or duration of exposure.

Conventional systems for delivering phototherapy to infants suffer from drawbacks including negative side effects on the newborn (e.g., hypothermia, dehydration, risk of eye damage, etc.), lack of portability, lack of comfortability, bulkiness of light source, low irradiance levels of light source, and/or limited light coverage on the infant's skin. For example, existing methods for administering phototherapy for treating hyperbilirubinemia in newborns include positioning the newborn, fully nude and wearing eye patches to protect the eyes, under lights (e.g., blue fluorescent light). Not only are these systems bulky and non-portable, but the blue fluorescent light source does not produce enough heat to the infant. Therefore, the infant can develop hypothermia. To prevent hypothermia, more fluorescent lights of other wavelengths can be added to the system to produce enough heat; however, this can lead to the infant becoming dehydrated. In phototherapy systems using other light sources, other challenges exist such as tradeoffs between adequate irradiance of the light panel, portability of the system, comfortability of the system, and light coverage. For example, phototherapy systems that have irradiance suitable for treating jaundice in infants may suffer from low coverage on the skin. Alternatively, phototherapy systems that are less bulky and/or are not stationary may not produce suitable irradiance levels.

In contrast, the embodiments described herein relate to a phototherapy system including a light panel configured to be removably disposed in a garment shell. In some embodiments, the garment can be a swaddle configured to receive an infant. The light panel may include a plurality of light sources (e.g., light emitting diodes (LEDs), organic light emitting diodes (OLEDs), fiber optic cables, etc.). The garment may be configured to position and/or orient the light panel over a portion of the infant's body. In some embodiments, the garment may include a pocket in which the light panel can be removably disposed. The pocket may include a transparent material such that light emitted from the panel can contact the skin of the infant. In some embodiments, the garment may include a second pocket configured to hold a power source (e.g., a portable and/or rechargeable battery) and/or a controller. In some embodiments, the garment shell may be configured to hold a portable power source and controller such that the phototherapy system is fully portable. In some embodiments, the phototherapy system described herein may allow the infant to breastfeed while receiving light therapy and promote parent-child bonding.

Embodiments described herein can include a light panel configured to produce a suitable level of irradiance for treating jaundice while also being light weight (e.g., less than about 12 ounces while having a coverage area of up to about 1300 cm² (centimeters squared), comfortable for the infant, and operational on levels of power that can be provided by a small battery pack to not disturb the infant. The phototherapy system described herein may include a light panel including a plurality of LEDs arranged in an ordered formation and coupled to a circuit board. The light panel and/or the circuit board may be disposed in a protective shell. The protective shell may include a plurality of cut outs (e.g., indentations, spaces, openings, through holes, pockets, etc.), which can reduce an overall weight of the light panel. In some embodiments, each of the plurality of cut outs can act as a convex lens to the skin. For example, a surface closer to the skin that defines the cut outs may be convex. The protective shell can protect the light panel and associated circuitry from being exposed to damaging materials such as liquid and/or protect the infant from the circuitry of the light panel. In some embodiments, the protective shell can act as a light guide, guiding light from the light source towards the skin of the infant, thereby reducing stray light (e.g., light that does not reach the infant's skin). In some embodiments, the protective shell may include, and the silicone may act as a light guide for light emitted from a light source.

Additionally, embodiments described herein relate to a phototherapy system including a controller configured to control light delivered by the light panel in response to at least one of user input or data collected. In some embodiments, the phototherapy system may include one or more sensors operatively coupled to the light panel and the controller. The controller may be configured to receive information including at least one of data from the sensors or a user input (e.g., from an external device) and adjust an output of the light panel in response to the information received. In some embodiments, the controller may be configured to lower an irradiance level of the light sources and/or turn off the light panel. In some embodiments, the controller may automatically (e.g., without user input) adjust the output of the light panel.

The phototherapy system described herein can use LEDs, OLEDs and/or fiberoptics for distributing the appropriate light over a large area of the infant's body. The phototherapy system is effective, inexpensive, lightweight, baby-friendly, durable, and easy to set up and use. The phototherapy system can consume relatively low power from a rechargeable battery and can be used outside of a hospital or during travel. The phototherapy system can minimize discomfort to babies and caregivers and can output light at one or more predetermined wavelength range for treating an associated condition (e.g., jaundice). The phototherapy system may include a real-time monitoring system (RTMS), a controller, a communication system, and one or more user interfaces (UIs). Thus, the phototherapy system can communicate with users and other devices and systems and enables quick response from the users (e.g., remotely). The phototherapy system delivers phototherapy that causes little to no side effects of hypothermia, dehydration, or rash etc. and requires no eye protector. The ability for the phototherapy system to provide suitable light therapy while having low power consumption enables the operation with a battery, so feeding and holding of the infant does not impact treatment, thereby promoting parent-child bonding.

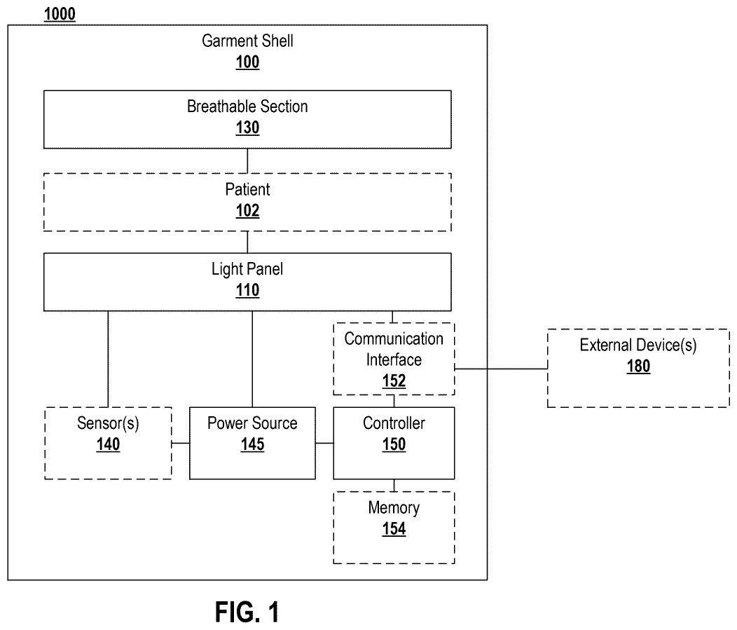

FIG. 1 is a schematic block diagram of a phototherapy system 1000 for delivering phototherapy to a patient 102, according to embodiments. As shown, the phototherapy system 1000 includes a garment shell 100 (e.g., a swaddle, gown, shirt, dress, vest, bib, etc.). In some embodiments, the garment 100 may be reusable and/or washable between uses. The garment shell 100 may be configured to be opened such that the patient 102 (e.g., an infant/newborn) can be disposed inside the garment shell 100. For example, the garment shell 100 may include a fastener (e.g., a zipper, VELCRO®, buttons, straps, clips, etc.) extending along a front side of the garment shell 100 and configured to open the garment shell 100 such that the patient 102 can be disposed inside and/or so that the garment shell 100 can be disposed around the patient 102. The fastener can include a two-way zipper which allows opening the garment 100 from the top and/or the bottom (e.g., for easy diaper change for infants without interrupting treatment). For example, the user may unzip from a top end (e.g., near the head of the infant) towards a bottom end (e.g., near the feet of the infant) of the garment shell 100 and/or unzip from the bottom end towards the top end of the garment shell 100.

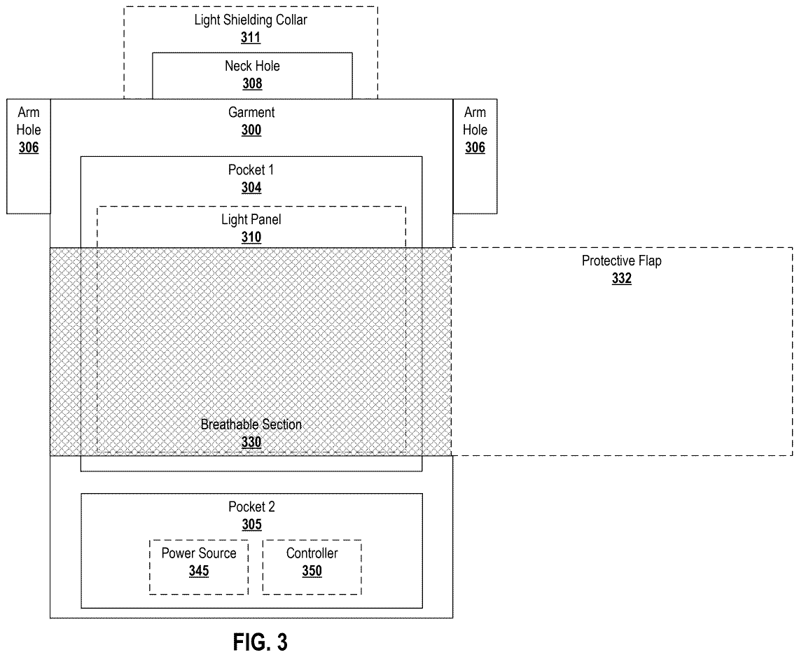

In some embodiments, the garment shell 100 includes a breathable section 130 disposed on and/or forming a portion thereof. In some embodiments, the breathable section 130 may include a breathable fabric such as, for example, a mesh, a fabric with openings, a lightweight fabric, etc. The breathable section 130 may be configured to allow a release of light and/or heat emitted by the light panel. In some embodiments, the garment shell 100 may further include an active heat dissipation mechanism such as a fan, blower, cooling material, etc. The active heat dissipation mechanism may be configured to move hot air out of the inside of the garment 100 and move cool air into the inside of the garment 100. In some embodiments, the garment 100 may optionally include a movable section (e.g., a movable flap). The movable flap may be attached to a side of the garment 100, for example. The movable flap may have a first configuration in which the movable flap does not cover the breathable section 130, and a second configuration in which the movable flap covers the breathable section 130 to block light emitted by the light panel 110 from existing the breathable section 130. The garment 100 may further includes a pair of arm holes and a neck hole such that the infant can wear the garment 100. In some embodiments, the bottom end of the garment 100 (e.g., the swaddle) may be closed. Further details of the garment 100 are described in FIG. 3 below.

The phototherapy system 1000 may include a light panel 110 (i.e., a light producing panel assembly (LPPA)) configured to be removably disposed in at least a portion of the garment shell 100. In some embodiments, the garment shell 100 may include one or more pockets on an inner surface and/or on an outer surface of thereof. For example, the garment shell 100 may include a first pocket on an inner surface thereof, the first pocket defining a space in which the light panel 110 may be at least temporarily disposed. The light panel 110 may be formed in or include a shape that corresponds to the space of the first pocket. In some embodiments, the first pocket may be configured such that the light panel 110 may be aligned over and/or covering a target area of the patient 102 when the light panel 110 is disposed in the first pocket. For example, the first pocket may be configured such that the light panel 110 is positioned over or near a portion of a torso of the patient 102 (e.g., a back and/or a chest of the patient 102). In some embodiments, the light panel 110 may be positioned to emit light on the upper body in 360 degrees and to emit light on a full back side of the lower body. In some embodiments, the light panel 110 and/or the garment 100 may be configured such that light emitted from the light panel 110 may cover or contact at least 1% to 100% of the infant's body. The light panel 110 may include a plurality of light sources (e.g., LEDs, OLEDs, fiber optics, etc.) configured to emit light towards the skin of the patient 102 when the light panel 110 is disposed in the garment shell 100 (e.g., the first pocket of the garment shell 100).

In some embodiments, the plurality of light sources may be in an ordered configuration (e.g., a grid, a staggered grid, a repetitive pattern, etc.). The plurality of light sources may be coupled to a circuit board configured to electrically connect (e.g., at the correct polarity) the light sources to the power supply 145 and/or to the controller 150. The circuit board may be configured to control output of the light sources 112 in response to signals from the power supply 145 and/or controller 150. In some embodiments, electrical connections of the phototherapy system 1000 may include heat dissipation components such as copper traces, copper coins and/or graphene film attached to one or both sides of the electrical connections or electrical circuits. In some embodiments, electrical connections in the light panel 110 may include the heat dissipation components. The electrical circuits of the phototherapy system 1000 may all include electrical connectors compatible with medical requirements for treatment.

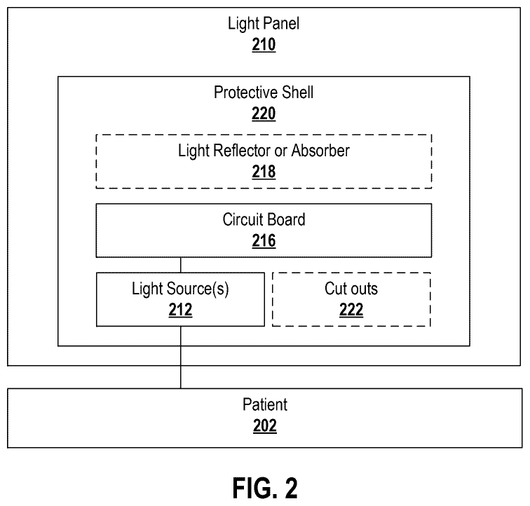

The light panel 110 may further include a protective shell configured to be disposed on and/or around a portion of the light sources 112 and the circuit board 116. The protective shell may be disposed on one or both sides of the light sources and circuit board and may be configured to protect the electronics from damaging materials and/or to protect the patient from the electronics. In some embodiments, the protective shell may include a plurality of cut outs. In some embodiments, the light panel 110 may be configured such that a space is maintained between the light panel 110 and a chest of the infant. For example, the light panel 110 may create a tent shape such that at least a portion of the light panel 110 is lifted off of the chest of the infant. Therefore, little to no weight from the light panel 110 may press on the chest of the infant, promoting more safe and comfortable conditions during the phototherapy session. Further details of the light panel 110 are described in FIG. 2 below.

The phototherapy system 1000 may further include the controller 150, the power source 145, and may optionally include one or more sensors 140, a communication interface 152, a memory 154, and one or more external devices 180. In some embodiments, the garment shell 100 may include one or more pockets defining a space configured to at least temporarily receive the controller 150 and/or the power source 145 (and/or the memory 154, the communication interface 152). For example, the garment shell 100 may include a second pocket defining a space for both the controller 150 and power source 145. In this way, the phototherapy system 1000 may be fully mobile and/or portable. In some embodiments, the garment shell 100 may include different pockets for the power source 145 and the controller 150. In some embodiments, the battery source 145, the controller 150, and the light panel 110 may all be disposed in the same pocket.

In some embodiments, the power source 145 may be operatively coupled to the light panel 110 and configured to provide power to the light panel 110. In some embodiments, the power source 145 may be any suitable power source such as a portable battery, a rechargeable battery, or a power cord coupled to a wall plug, for example. In some embodiments, the power source 145 may be a portable and rechargeable battery. The power source 145 may be light weight and low volume such that the power source 145 fits comfortably in the garment shell 100 and does not disturb the patient 102 while worn. In some embodiments, the power source 145 may be configured to provide constant power to the phototherapy system 1000. In some embodiments, the power source 145 may be a battery couplable to a cord and wall plug that may be configured to provide constant power to the phototherapy system 1000. In some embodiments, the power source may be a battery couplable to a cord and wall plug, the battery configured to provide power to the phototherapy system 1000 when not connected to the wall plug. In some embodiments, the power source 145 may be a rechargeable battery, and the rechargeable battery may provide between about 1 hour to about 3 hours of power when fully charged. In some embodiments, the power source 145 may provide power that is about equal to three times a length of an average breastfeeding time for the infant.

The sensor(s) 140 may be configured to monitor at least one of a biosignal of the patient 102 and/or a signal related to an environment inside the garment 100. For example, the sensor(s) 140 may be configured to monitor at least one of a location of the garment 100, a temperature inside the garment 100, a temperature of the light panel 110, a humidity inside the garment, and/or a bilirubin level of the patient 102. The sensor(s) 140 can include at least one of a temperature sensor, a humidity sensor, wetness sensor, a light intensity sensor, a global position system (GPS) sensor, a sound detector, a motion detector, an accelerometer, and/or a bilirubin meter. In some embodiments, the signals the sensor(s) 140 collect may be safety parameters to promote safe operating of the phototherapy system 100 for treating an infant. In some embodiments, the sensor(s) 140 may be included in the light panel 110. In some embodiments, the sensor(s) 140 may be integrated into the garment 100 (e.g., sewn into a portion of the garment 100). In some embodiments, the sensor(s) 140 may be integrated into a circuit board (e.g., the FCB 216) of the light panel 110. In some embodiments, the sensor(s) 140 may be configured to be coupled to a skin of the patient 102. In some embodiments, the sensor(s) 140 may communicate the data collected to the controller 150 for analysis, storage, and/or communication to the external device 180. In some embodiments, the sensor(s) may be configured to continuously monitor the signals. In some embodiments, the sensor(s) may be configured to monitor the signals periodically and/or at predetermined time intervals.

In some embodiments, the controller 150 may be operably coupled to the light panel 110, the power source 145, the sensor(s) 140, and/or the external device(s) 180. The controller 150 may be configured to receive information associated with a light therapy session. For example, the controller 150 may receive information from at least one of the external device(s) 180, the sensor(s) 140, and/or directly from the light panel 110. The information can include, but is not limited to, a biosignal of the patient 102 such as bilirubin levels (e.g., collected by the sensor(s) 140), patient history of the patient 102, user input (e.g., from the external device(s) 180), output parameters of the light panel, a duration of time the light panel has been active, and/or a prescription (e.g., time and intensity of light) for the light therapy. The controller 150 may be configured to adjust an output of the light panel 110 based on the information received. In some embodiments, the controller 150 may adjust the irradiance of the light sources 110 by changing a current delivered to the light panel 110. In some embodiments, the information the controller 150 monitors may correspond to the one or more safety parameters for delivery safe phototherapy to infants. In some embodiments, the controller 150 may be configured to detect when the one or more safety parameters crosses a predetermined threshold and/or falls outside of a predetermined range. In some embodiments, a safety parameter may have a predetermined range and if the measured signal falls outside of that range, the controller 150 may adjust the output of the light panel 110, and/or alert (e.g., using sounds, text messages, flashing light, buzzing, instructions, dialogue windows, etc.) the user via the external device 180. In some embodiments, the controller 150 may cause the external device 180 to display a message informing the user that the light panel 110 has been automatically shut off and/or an intensity of the light source has been reduced. In some embodiments, the controller 150 may alert and/or display a message to the user when switching on (e.g., automatically) the light panel 110 based on the parameters set by the user (e.g., on the external device 180). In some embodiments, the controller 150 can turn off (e.g., automatically) the light panel 110 based on treatment cycles the user has input on a user interface (e.g., on the external device 180). In some embodiments, the user can also configure the phototherapy system 1000 to only send alerts and display messages, so the users can manually turn on and/or off the light panel 110. In some embodiments, the controller 150 may be configured to cause the external device 180 to alert the user when a power level of the phototherapy system 1000 is low.

In some embodiments, a minimum threshold of the intensity of light from the light sources 112 at the surface of the skin of the patient 102 may be at least about 15 μW/cm²/nm (microwatts per centimeter square per nanometer) or at least about 30 μW/cm²/nm. In some embodiments, a predetermined upper threshold of a temperature level inside the garment 100 is about 40 degrees Celsius (C). In some embodiments, a predetermined upper threshold of a temperature emitted by the light panel 110 is between about 38 degrees C. and about 40 degrees C. In some embodiments, a predetermined range of humidity level inside the garment 100 is between about 10% and about 90%, inclusive of all ranges and subranges therebetween. In some embodiments, a duration of time of the phototherapy session may be in a range of about 5 minutes to about 24 hours, inclusive of all ranges and subranges therebetween. In some embodiments, the duration of time of the phototherapy session may be in a range of about 45 minutes to about 3 hours, inclusive of all ranges and subranges therebetween. In some embodiments, the duration of the phototherapy session may correspond to an amount of time the infant is normally fed such that the phototherapy session can be completed during feeding. In some embodiments, the duration of the phototherapy session may depend on user input and/or a prescription.

In some embodiments, the controller 150 may be configured to receive, from the external device 180, information associated with the light therapy session (e.g., a duration of the session, an intensity and/or wavelength of the light for session, one or more safety parameter ranges, etc.). The controller 150 may be configured to send a signal to the light panel 110 to cause the light panel 110 to emit light based on the information associated with the light therapy session. In some embodiments, the controller 150 may be configured to monitor, via the sensor(s) 140, one or more signals including at least one of the biosignal of the patient 102 or a signal related to the environment inside the garment 100. In some embodiments, the controller 150 may be configured to determine or detect whether the one or more signals crosses a predetermined threshold and/or falls outside of a predetermined range. The controller 150 may be configured to adjust the output of the light panel 110 in response to at least one of the one or more signals crossing the predetermined threshold and/or falling outside of the predetermined range. For example, the controller 150 may drop the light intensity output from the light panel 110 to below a certain value in response to a signal crossing the predetermined threshold. In some embodiments, the controller 150 and/or a circuit board coupled to the light panel 110 may include electrical circuits to power the light panel 110 according to one or more light panel parameters such as for example, constant voltage and/or pulsed at a higher voltage, frequencies, and/or duty cycles to produce a desired light output.

The controller 150 may include one or more processors, and the processors may be configured to carry out functions, processes, and/or modules for operation of the phototherapy system 1000. The processor(s) of the controller 150 can be any suitable processing device(s) configured to run and/or execute a set of instructions or code. For example, the processors can be and/or can include one or more data processors, image processors, physics processing units, digital signal processors (DSP), analog signal processors, mixed-signal processors, machine learning processors, finite state machines (FSM), compression processors (e.g., data compression to reduce data rate and/or memory requirements), encryption processors (e.g., for secure wireless data and/or power transfer), and/or the like. The processors can be, for example, a general-purpose processor, central processing unit (CPU), microprocessor, microcontroller, Field Programmable Gate Array (FPGA), an Application Specific Integrated Circuit (ASIC), a processor board, a virtual processor, and/or the like. The processors can be configured to run and/or execute or implement software application processes and/or other modules, processes and/or functions related to controlling the output of the light panel 110 and/or monitoring biosignals of the patient 102. The underlying device technologies may be provided in a variety of component types, for example, metal-oxide semiconductor field-effect transistor (MOSFET) technologies like complementary metal-oxide semiconductor (CMOS), bipolar technologies like generative adversarial network (GAN), polymer technologies (e.g., silicon-conjugated polymer and metal-conjugated polymer-metal structures), mixed analog and digital, and/or the like.

In some embodiments, the controller 150 may be coupled to a memory 154 for storing information (e.g., information related to the phototherapy session such as a duration of the session, the bioisgnals, and/or information about an environment of the phototherapy system 1000). Additionally or alternatively, the controller 150 may be configured to send all information to the external device(s) 180 for storage. In other words, the controller 150 may not store information onboard. The memory 154 can be any suitable memory device(s) configured to store data, information, computer code or instructions (such as those described herein), and/or the like. The memory may store (1) phototherapy session information such as duration, intensity, patient identifying information, etc.); (2) data collected from the sensor(s); (3) and/or any other information related to functioning of the system 1000. In some embodiments, the memory can be and/or can include one or more of a random access memory (RAM), static RAM (SRAM), dynamic RAM (DRAM), a memory buffer, an erasable programmable read-only memory (EPROM), an electrically erasable read-only memory (EEPROM), a read-only memory (ROM), flash memory, volatile memory, non-volatile memory, combinations thereof, and the like. In some embodiments, the memory may also be configured to at least temporarily store sensor(s) data, for example, until the data is transmitted to the external device(s) 180.

In some embodiments, the controller 150 may be configured to send information related to the light panel 110, the power source 145, and/or the sensor(s) 140 to the external device(s) 180. In some embodiments, at least one of the controller 150 and the external device(s) 180 may be configured to process and/or analyze data such as, for example, biosginal data, location data, temperature data, and/or humidity data. In some embodiments, the controller 150 may process and/or analyze the data and in real-time or near real-time and control the light panel 110 in response to the processed and/or analyzed data. For example, the controller 150 may receive sensor data including a raw signal associated with a bilirubin level of the patient 102, and the controller 150 may be configured to process and/or analyze the raw signal and determine the bilirubin level of the patient 102. The processor 150 may then reduce and/or increase an intensity of the light output from the light panel 110 based on the bilirubin level. In some embodiments, the processor 150 may turn off and/or stop (e.g., automatically or without user input) the light panel from outputting light based on at least one of the sensor data (e.g., bilirubin levels), a user input, and/or a prescription for the light therapy. In some embodiments, the external device 180 may be configured to at least partially process and/or analyze sensor data for the sensor(s) 140 and send the processed data to the controller 150 to control the light panel 110.

In some embodiments, the controller 150 and/or the power source 145 may include a display configured to display information to the user. In some embodiments, the controller 150 and/or the power source 145 may include one or more input/output (I/O) devices configured to receive user inputs directly from the user.

The external device 180 can include any suitable compute device such as, for example, one or more user device(s), a physician device, a server, and/or a database. The external device 180 may include a phone, a tablet, a laptop computer, a desktop computer, or any other suitable compute device. In some embodiments, the external device(s) 180 may include a user interface configured to display information related to the phototherapy sessions to the user and/or receive user input for controlling the led panel 110. In some embodiments, the external device(s) 180 may include a display such that the controller 150 can cause the external device(s) 180 to display information related to the phototherapy and/or the patient. For example, the controller 150 may be configured to send information related to an intensity and/or wavelength of the light output from the light panel 110 such that the display displays this information to the user. In some embodiments, the external device 180 may be configured to run an application on a touch screen, a website running remotely, and/or execute remote procedure call services. In some embodiments, the external device 180 (e.g., via the user interface) may be configured to display the monitoring information, alert users, and take the commands from the users to control the light panel 110 operation status and set up light panel parameters.

In some embodiments, users can be locally and/or remotely connected to the phototherapy system 1000. For example, the controller 150 and the external device(s) 180 may be configured to communicate remotely. For example, the phototherapy system 1000 may include the communication interface 152 configured to communicate wirelessly (e.g., via Bluetooth, Wi-Fi, cellular such as 3G, 4G, 5G, etc., 802.11X Zigbee, etc.). In some embodiments, the communication interface 152 can include one or more satellite, WI-FI, Bluetooth, or cellular antenna). In some embodiments, the communication interface 152 may include one or more senders, receivers, and/or double-direction transportation channels. The senders and the receivers can exchange data through the double-direction transportation channels. In some embodiments, the senders can be receivers and the receivers can be senders. In some embodiments, the senders and the receivers can be anything the transportation channels connect to, including the light panel 110, the controller 150, the external device 180, and/or a data server or other medical devices. In some embodiments, the communication interface 152 may be modularized to allow easy extension of new features. The communication interface 152 may be integrated into the controller 150 and/or a separate hardware component. In some embodiments, the communication interface 152 may communicate some or all information between the controller 150 and the light panel 110 and/or the controller 150 and the external device 180.

FIG. 2 is a schematic block diagram of a light panel 210 for delivering light to skin of a patient 202, according to embodiments. In some embodiments, the light panel 210 may include a plurality of light sources 212 (e.g., LEDs, OLEDs, fiber optics) coupled to a circuit board (e.g., a flexible circuit board (FCB)) 216. In some embodiments, the light source(s) 212 may be arranged in an ordered configuration (e.g., a grid configuration, a zig-zag configuration, a repetitive pattern, etc.). In some embodiments, the light sources 212 (e.g., when disposed in a pocket of the garment) may be configured to be disposed near a surface of the skin of the patient 202 and emit light on the surface of the skin of the patient 202. In some embodiments, the light sources 212 (e.g., LEDs) may have a viewing angle and/or be spaced from one another such that an intensity of light emitted from the light sources 212 is above a predetermined threshold at the surface of the skin of the patient 202. In some embodiments, the light sources 212 may be spaced from one another such that no gaps in illumination exist at a side of the protective shell 220 closest to the patient 202 and/or on the surface of the skin of the patient 202. For example, the light source 212 can be LEDs that are set apart (e.g., spaced) from one another such that the light projected by each LED on a surface that is a predefined distance away overlaps or extends from the light projected by its adjacent LEDs on that surface. In the case of LEDs having predefined viewing angles, the LEDs can be set at a predetermined distance from one another based on the viewing angle and a predetermined distance to the surface. In some embodiments, the viewing angle and the distance between the light sources 212 may be configured such that a light intensity is substantially evenly distributed across the surface of the skin of the patient 202.

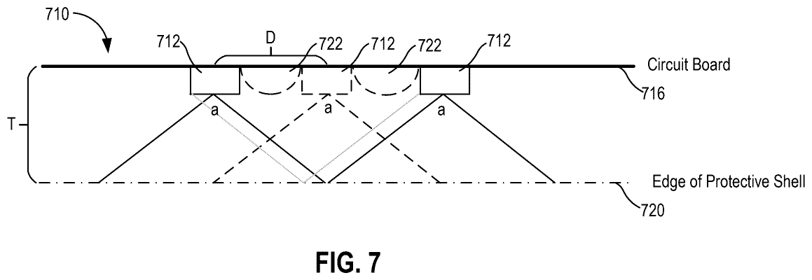

In some embodiments, the light panel 210 may include about 10 light sources to about 2000 light sources, inclusive of all ranges and subranges therebetween. In some embodiments, the viewing angle of each light source 212 may be in a range of about 0 degrees to about 180 degrees, inclusive of all ranges and subranges therebetween. In some embodiments, the viewing angle of each light source 212 may be in a range of about 60 degrees to about 130 degrees, inclusive of all ranges and subranges therebetween. In some embodiments, it may be desirable to have a larger viewing angle such that each light source can project light over a larger area, and so that less light sources may need to be used to project light over a larger surface area. In some embodiments, a distance between adjacent light sources 212 may be in a range of about 2 mm to about 20 mm, inclusive of all ranges and subranges therebetween. As described above, in some embodiments, this distance may be set based on the viewing angle of the light sources 210 and/or a predetermined distance to a surface that the light is projected toward. Additionally, as described below, the light sources 210 may be disposed in a protective shell 220. When disposed in a protective shell 220, the shell may allow for transmission of light, such that light projected by the light sources 210 can reach a target surface (e.g., the skin of an infant). Additionally, to ensure that light is distributed across the skin of the infant, it may be desirable to have light sources 210 that collectively can project light across the thickness of the protective shell 220 to each point of the outer surface or edge of the protective shell 220. The light that projects outward from the edge of the protective shell 220 can then be configured to provide an even distribution of light. Further details of such an arrangement are described with respect to FIG. 7.

The plurality of light sources 212 and/or the FCB 212 may be disposed in a protective shell 220. In some embodiments, the protective shell 220 may include one or more portions or layers. In some embodiments, the protective shell may include single or multiple protection layers that cover the light sources 212 of the panel to protect the light sources 212 from fire and fluids such as urine, food, feces, etc. In some embodiments, the protective shell 220 may be waterproof or liquid proof. In some embodiments, the protective shell 220 may include a first portion or layer configured to be disposed on a first side of the light source(s) 212 (e.g., the light emitting side) and/or a first side of the FCB 216. The first portion or layer of the protective shell 220 may protect the light source(s) 212 from damage (e.g., liquids such as sweat or urine or physical damage from force applied to the light source(s) 212). In some embodiments, the protective shell 220 may include a second portion or layer configured to be disposed on a second side of the light sources 212 (e.g., the non-emitting side) and/or a second side of the FCB 216 such that the protective shell 220 envelopes the light source(s) 212 and/or the FCB 216. In some embodiments, the first portion may be joined (e.g., sealed, affixed) to the second portion to form the shell to protect an internal environment of the shell from the external environment. In some embodiments, the protective shell 220 may act as a light guide to guide light from the light source(s) 212 to the skin of the patient 202. In some embodiment, the protective layer 220 may reduce stray light, e.g., by having surfaces that block light from straying outside of the desired target area or reflect light back toward the desired target area.

The protective shell 220 may include a transparent material such that light can pass through the protective shell 220 towards the patient 202. In some embodiments, at least a portion of the protective shell 220 covering the light sources 212 (e.g., a side of the light sources facing the patient 202) may have transparent material. In some embodiments, the protective shell 220 may be flexible such that the protective shell 220 can at least partially conform to the garment and/or the body of the patient 220. In some embodiments, the protective shell 220 can include any suitable material such as, for example, silicone, polyvinyl chloride (PVC), high medical grade transparent silicone, or a suitable combination thereof. In some embodiments, a protection level of the protective shell 220 material may be IP65. The material of the protective shell 220 may be a medical grade material. In some embodiments, the protective shell 220 may include materials which are compatible with standard cleaning and disinfectant fluids.

In some embodiments, the protective shell 220 may include a light-reflective layer 218, the light-reflective layer disposed on the second side of the light sources 212 and/or the second side of the FCB 216 such that stray light from the plurality of light sources 212 is reflected back to the surface of the skin of the patient. In some embodiments, the protective shell 200 may include a light-absorbing layer 218, the light-absorbing layer disposed on the second side of the FCB 216 such that stray light from the plurality of light sources 212 is absorbed. In some embodiments, the light-absorbing layer may include an opaque material that absorbs light. In some embodiments, the light-absorbing layer may include a material that is dark in color (e.g., dark blue, black, etc.). In some embodiments, the light-reflective layer may include a reflective material (e.g., silver/chrome).

In some embodiments, the protective shell 220 may include a plurality of cut outs 222 (e.g., indentations, spaces, openings, through holes, pockets, etc.). For example, the protective shell 200 may define the plurality of cut outs 222 between at least some adjacent light sources 212 of the plurality of light sources 212. In some embodiments, the first layer of the protective shell 220 may define a first portion of the cut outs 222 and the second layer of the protective shell 220 may define a second portion of the cut outs 222. In some embodiments, the first portion and the second portion may be configured to align to define an inner volume when the first layer and the second layer are adjoined. In some embodiments, only the first layer and/or only the second layer of the protective layer may define cut outs 222. In some embodiments, the cut outs 222 may be through holes (e.g., an absence of material) that extend from a first surface of the protective shell 220 to a second surface of the protective shell 220 such that the protective shell 220 is openwork or forms a lattice structure. In some embodiments, the protective shell 220 may include a plurality of indentations. In some embodiments, the cut outs 222 may be in one or more layers of the protective shell 220.

In some embodiments, the cut outs 222 may include any suitable shape such as, for example, a circle, an oval, a square, a rectangle, a diamond, a polygon, an abnormal shape, etc. In some embodiments, the indentations form an oval (e.g., an ellipsoid or a semi-ellipsoid).

In some embodiments, a total thickness of the protective shell 220 may be in a range of about 2 mm (millimeters) to about 10 mm, inclusive of all ranges and subranges therebetween. In some embodiments, a total thickness of each cut out 222 may be in a range of about 0.5 mm to about 9.5 mm, inclusive of all ranges and subranges therebetween. In some embodiments, the cut outs 222 may define an inner volume, and the inner volume of the cut outs 222 may be in a range of about 0.5 mm³ to about 5 cm³, inclusive of all ranges and subranges therebetween. In some embodiments, a maximum cross-sectional area of the cut outs 222 may be in range of about 0.5 mm² to about 15 cm², inclusive of all ranges and subranges therebetween. In some embodiments, a total weight of the protective shell 220 with the cut outs 222 may be in a range of about 0.1 g (grams) to about 1 kg (kilogram), inclusive of all ranges and subranges therebetween. In some embodiments, the cut outs 222 may reduce a total weight of the protective shell 220 up to about 50%, about 60%, about 70%, about 80%, about 90%, about 95%. In some embodiments, the cut outs 222 may reduce a total weight of the protective shell 220 by about 95%. In some embodiments, the cut outs 222 may reduce a total weight of the light panel 210 by about X %. The reduced weight of the light panel 210 may reduce a pressure or force the light panel 210 exerts on a chest of an infant.

In some embodiments the cut outs 222 may be disposed over the light sources 212 such that light passes through the cut outs 222. In some embodiments, the cut outs 222 may be disposed between, adjacent, and/or above the light sources 212 such that the cut outs 222 are positioned outside of a light cone defined by the viewing angle of the light sources 212 and/or other path of light of the light sources. Alternatively, the cut outs 222 may intervene with a light path of the light sources 212, thereby changing a light distribution of light emitted from each light source 212. In some embodiments, the cut outs 222 may act like a converging lens. In some embodiments, the inner volume of the cut outs 222 may have a different refractive index than a refractive index of the protective shell material such an angle of light changes as the light moves through the cut outs 222. In some embodiments, the cut outs 222 (e.g., indentations) may be fully contained in the protective shell 220 meaning that an outer surface of the protective shell 220 feels smooth.

In some embodiments, the light panel 210 may be configured to emit light in a range of 0 μW/cm²/nm to about 100 μW/cm²/nm, inclusive of all ranges and subranges therebetween. In some embodiments, the light panel 210 may emit light at least 30 μW/cm²/nm across an entire surface area of the light panel 210. In some embodiments, a wavelength of light emitted from the light panel 210 may be in a range of about 450 nm to about 500 nm, inclusive of all ranges and subranges therebetween. In some embodiments, a wavelength of light emitted from the light panel 210 may be in a range of about 465 nm to about 475 nm, inclusive of all ranges and subranges therebetween. In some embodiments, a surface area (e.g., an area of the patient) that the light panel 210 may be configured to emit light onto may be about 10 cm², 50 cm², 100 cm², 200 cm², 300 cm², 400 cm², 500 cm², 600 cm², 700 cm², 800 cm², 900 cm², 1000 cm², 1100 cm², 1200 cm², 1300 cm², 1400 cm², 1500 cm², 1600 cm², 1700 cm², 1800 cm², 1900 cm². In some embodiments, the surface area may be up to 1300 cm². In some embodiments, the light panel 210 may be configured to run using between about 5 mW to about 100 mW of power. In some embodiments, the light panel 210 may be configured to run using between about 50 mW to about 100 mW of power. In some embodiments, the light panel 210 may have about 10,000 to about 100,000 hours of lifetime.

In some embodiments, the light panel 210 may have any suitable configuration. For example, the light panel 210 may include a fiberoptic light source panel made of a single piece or assembled by several pieces to distribute the light. In some embodiments, the fiberoptic light source panel may be a flexible woven fiberoptic light source panel. In such embodiments, the fiberoptic panel may include a single layer or multiple layers, which can be assembled from a single piece or assembled from several pieces, to distribute the light. In some embodiments, the light panel 210 may include a plurality of LEDS couple to a flexible fiberoptic panel (FFP) to produce light with a predetermined wavelength and intensity and distribute light over a large surface area (e.g., greater than 1000 cm²). In some embodiments, the LEDs may be the light sources of the optical fibers which are coupled to the LEDs. In some embodiments, the optical fibers may be disposed in plastic shells. In some embodiments, the LED light sources may be disposed outside of the garment shell. In some embodiments, the LEDs may be configured to produce light in opposing directions at opposite sides of the fiberoptic segment to increase light intensity. In some embodiments, the light panel 210 may include a plurality of pixels assembled by several LEDs. In some embodiments, the LEDs may be positioned to form a shape integrated into a single piece with or without a fiberoptic panel. In some embodiments, the light panel 210 may include a plurality of OLEDs and/or multiple pixels assembled by several OLEDs in any shape. The light panel 210 may be integrated into one piece with or without a fiberoptic panel. In some embodiments, the light panel 210 may include a plurality of portions coupled together to produce light with a predetermined wavelength and intensity and to distribute light over a large surface area. In some embodiments, the light panel 210 can be configured to include flexible arrays, meshes, sheets, or discrete devices. The light panel 210 may be structurally and/or functionally similar to the light panel 110, and therefore, certain details of the light panel 210 are not described in FIG. 2.

FIG. 3 is a schematic block diagram of a garment shell 300 configured to hold a light panel 310 for delivering phototherapy to an infant wearing the garment shell, according to embodiments. In some embodiments, the garment shell 300 may include a first fastener (e.g., a zipper) extending along the front side of the garment shell 300. The first fastener may be configured to be manipulated (e.g., unzipped, unclipped, unbuttoned, etc.) to open the garment shell 300 such that the infant can be disposed inside the garment shell 300. In some embodiments, the garment shell 300 may include any suitable material such as cotton, wool, polyester, linen, etc. In some embodiments, the garment shell 300 may include cotton. In some embodiments, a fabric of at least a portion of the garment shell 300 may be double-layered (e.g., to more effectively prevent light from escaping through the fabric than single layer). In some embodiments, at least a portion of the garment shell 300 may include double-layered cotton. In some embodiments, the garment shell 300 may be configured as one size or may be come in different sizes to fit to all body sizes and/or different age infants. In some embodiments, the garment shell 300 may be a swaddle. The garment shell 300, light panel 310, and breathable section 330 may be structurally and/or functionally similar to the garment shell 100, light panel 110, 210, and/or breathable section 130, and therefore, certain details of the garment shell 300 are not described in FIG. 3.

As shown, the garment shell 300 may include a first pocket (e.g., pocket 1) 304 disposed on or sewn to an inner surface of the garment shell 300. The first pocket may define a space in which a light panel is configured to be removably disposed. In some embodiments, the space of the first pocket may extend from a backside of the garment shell 300 to a front side of the garment shell 300. In some embodiments, the space may be configured such that the light panel covers a back of the infant and/or a portion of a front of the infant. In some embodiments, a first portion of the light panel 310 may cover a portion of the back of the infant and a second portion of the light panel 310 may cover a portion of the chest of the infant. In some embodiments, the first pocket 304 may include a transparent material such that light from the light panel 310 can shine through the transparent material towards the infant. In some embodiments, the first pocket 304 may include a mesh material. In some embodiments, the first pocket 304 may include a fabric such as polyester. In some embodiments, the first pocket 304 may include a second fastener (e.g., a zipper). In some embodiments, the second fastener may be accessible from a front and/or a back side of the garment 300. In some embodiments, the second fastener may be accessible from the back side of the garment 300. In some embodiments, the second fastener may be configured to be opened (e.g., unzipped) such that the light panel 310 can be disposed in the space.

In some embodiments, the garment shell 300 may include a second pocket (e.g., pocket 2) 305 defining a space configured to receive one or more additional components of the phototherapy system. For example, the second pocket 305 may be configured to receive the controller 350, the power source 345, and/or at least some of the sensor(s) 340. In some embodiments, the second pocket 305 may include a third fastener (e.g., a zipper) configured to open and close the second pocket 305. In some embodiments, the second pocket 305 may be disposed on a back side of the garment shell 300 and/or near a bottom of the garment shell 300.

In some embodiments, the garment shell 300 may further include a breathable section 330 (e.g., a mesh, lace, breathable fabric, one or more openings defined in a fabric, etc.) on at least a portion of the garment shell 300. The breathable section 330 may allow light or heat that is emitted by the light panel to exit the garment shell 300. In some embodiments, the breathable section 330 may at least partially align with a stomach and/or a bottom half of the infant when the infant is in the garment shell 300. In some embodiments, the breathable section 330 may be configured to align with at least a portion of the light panel 310 when the light panel 310 is disposed in the garment shell 300. In some embodiments, the garment shell 300 may include an active heat dissipation mechanism (e.g., a fan, a blower, a cooling material, etc.).

In some embodiments, the garment shell 300 may further include a protective flap 332. The protective flap 332 may be configured to selectively cover the breathable section 330. For example, the protective flap 332 may be movable between a first configuration in which the protective flap 332 does not cover the breathable portion 330, and a second configuration in which the protective flap 332 covers the breathable portion 330. In some embodiments, the protective flap 332 may be moved by a user (e.g., mother) between the first configuration and the second configuration. In some embodiments, the protective flap 332 may be coupled to and/or sewn to on side of the garment shell 300 such that the protective flap 332 can be moved over the breathable section 330. In some embodiments, the protective flap 332 may prevent light from escaping the garment shell 300. In some embodiments, the protective flap 332 may prevent light from shining toward the user holding the infant. In some embodiments, the protective flap 332 may include a material such as cotton (e.g., double-layered cotton). In some embodiments, the garment shell 300 may include a main body, the breathable section 330, and the protective flap 332. The main body and the protective flap 332 may include a first material and the breathable section 330 may include a second material that is different from the first material. In some embodiments, the first material may include cotton (e.g., double-layered cotton) and the second material may include a polyester (e.g., a mesh polyester). In some embodiments, the main body of the garment shell 300 may define a first space in which the infant is disposed and the first pocket 304 may define a second space separate from the first space in which the light panel is disposed.

In some embodiments, the garment shell 300 may further include a pair of arm holes 306. The pair of arm holes may define openings that are adjustable in size. For example, the arm holes 306 may each include zippers configured to loosen and/or tighten the arm holes 306. In some embodiments, the arm holes 306 may be configured to fit snug around the arms of the infant such that light does not escape the garment shell 300. For example, an outer edge of each arm hole 306 may be configured to abut the arm of the infant to prevent light from escaping through the arm hole 306. In some embodiments, the garment shell 300 may further include a neck hole 308 through which the infant's head may be disposed. In some embodiments, the neck hole 308 may be coupled to a light shielding collar (hereinafter, “collar”) 311 configured to prevent light from escaping the garment shell 300 through the neck hole 308. In some embodiments, the collar 311 may protect the eyes and/or brain of the infant from being exposed to light. In some embodiments, the collar 311 may allow the infant to not wear protective glasses during phototherapy. In some embodiments, the collar may include an adjustable member such as, for example, a drawstring, a VELCRO®, an elastic band etc. to seal the light inside the garment shell 300.

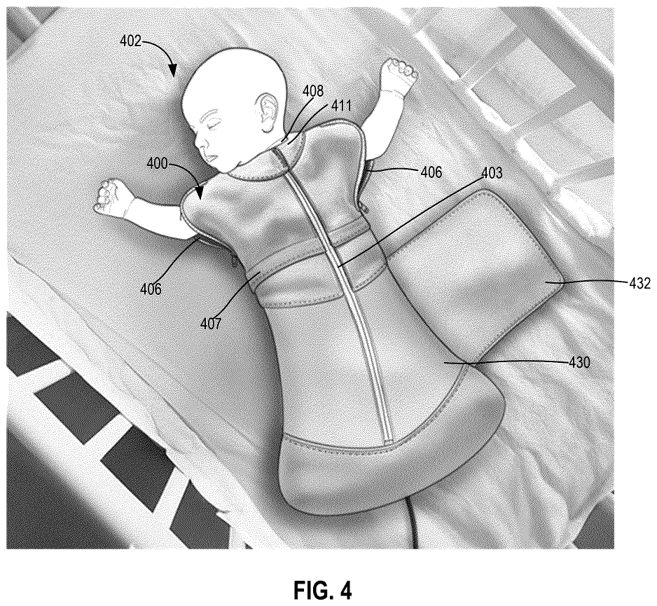

FIG. 4 shows a phototherapy system including a garment shell 400 worn by an infant 402 and configured to hold a light panel to provide phototherapy to the infant 402, according to embodiments. As shown, the garment shell 400 may be a swaddle including a closed bottom. The garment shell 400 may include a pair of arm holes 406 through which the infant's 402 arms may be disposed. Each arm hole 406 includes an adjustable portion (e.g., a zipper, drawstring, VELCRO®, etc.) such that the arm holes 406 can be adjusted to fit snug around the infant's arms and prevent light from escaping the garment shell 400 through the arm holes 406. As shown, the arm holes 406 may each be zipped along a length of the arm holes 406 to decrease and/or increase a diameter of an opening defined by the arm hole 406. The garment shell 400 may further include a neck hole 408 through which the infant's 402 head can be disposed. The neck hole 408 is coupled to a collar 411 including an adjustable portion such that the collar 411 can adjusted to prevent light from escaping the garment shell 400 and shining towards the infant's face.

The garment shell 400 includes a first fastener 403 (e.g., zipper) extending along a length of the garment shell 400 and configured to open the garment shell 400 such that the infant 402 can be disposed inside. In some embodiments, the first fastener 403 may be on a front side of the garment shell 400. In some embodiments, the first fastener 403 may be on a back side of the garment shell 400. The garment shell 400 may further include a second fastener 407 (e.g., zipper) extending around at least a portion of a torso of the infant 402. The second fastener 407 may be coupled to a first pocket (not shown) disposed inside the garment shell 400. The second fastener 407 may be configured to open the first pocket such that a user can access the first pocket and dispose a light panel for delivering photo therapy into the first pocket.

As shown, the garment shell 400 further includes a breathable section 430 configured to allow light and/or heat to move out of the garment shell 400 and/or to allow cool air to move into the garment shell 400. The breathable section 430 may cover a lower half of the front side of the infant 402. The garment shell 400 may include a protective flap 432 configured to be moved to cover at least a portion of the breathable section 430. As shown, the protective flap 432 is in an open configuration (e.g., a first configuration) and can be transitioned to the closed configuration by the user. The phototherapy system and garment shell 400 may be structurally and/or functionally similar to the phototherapy system 1000 and garment shells 100, 300, and therefore certain aspects of the garment shell 400 are not described herein.

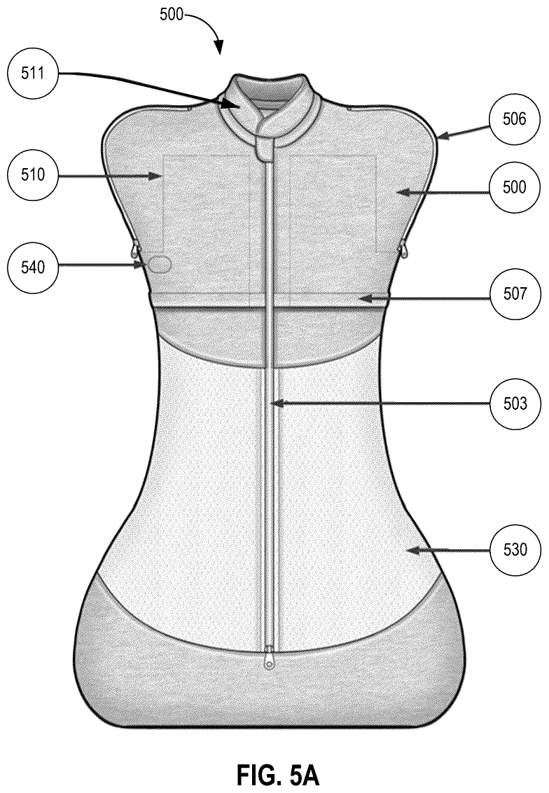

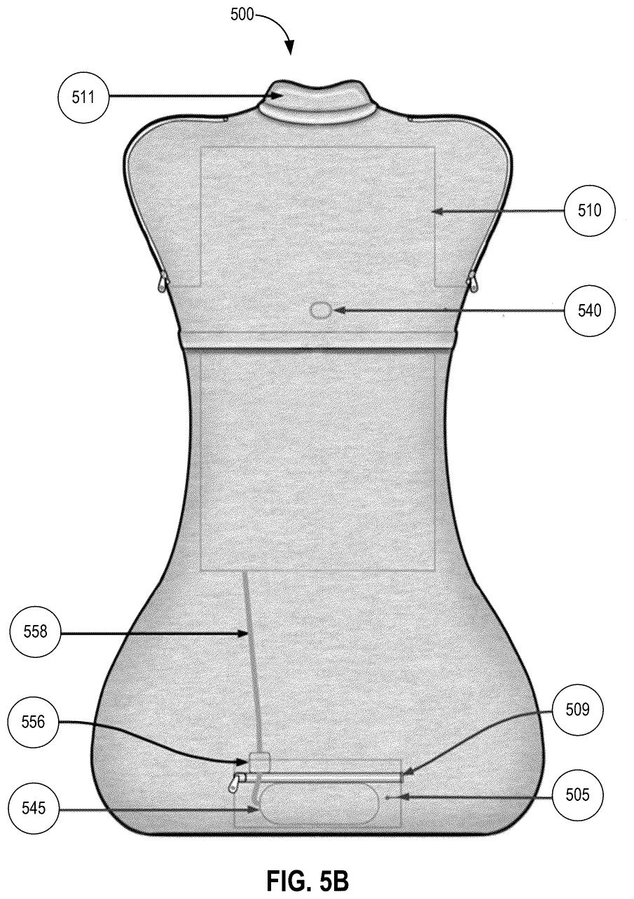

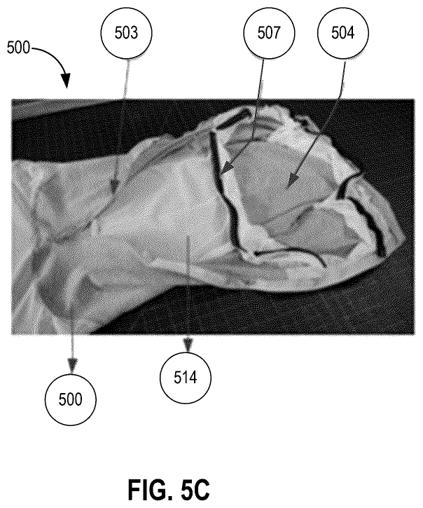

FIGS. 5A-5B show a front view and a back view, respectively, of a garment shell 500 configured to hold a light panel for delivery phototherapy to an infant, according to embodiments. As shown in FIG. 5A, the garment shell 500 includes a first fastener 503, a second fastener 507 coupled to a first pocket (not shown), a breathable section 530, a pair of arm holes 506, and a neck hole coupled to a collar 511. The garment shell 500 may be structurally and/or functionally similar to the garment shells 100, 300, 400, and therefore certain details of the garment shell 500 are not described herein.

The garment shell 500 may include sensor(s) 540 coupled to and/or sewn into a portion of the garment shell 500. As shown, the sensor(s) 540 is disposed on a chest portion of the garment shell 500 and/or a on a back portion of the garment shell 500. FIGS. 5A-5B shows an outline 510 corresponding to an upper boundary of the light panel inside a first pocket disposed in the garment shell 500. As shown, the light panel is configured to cover at least a chest of the infant.

As shown in FIG. 5B, the garment shell 500 further includes a second pocket 505 disposed on the back side of the garment shell 500. The second pocket 505 includes a zipper 509 configured to open and/or close the second pocket 505. The second pocket may be configured to receive the controller and/or the power supply 545. The power supply 545 may be configured to supply power to the light panel via one or more electrical connectors 556, 558 (e.g., a cable, wire, conductive trace, conductive thread, adapters, outlets, etc.). In some embodiments, the one or more electrical connectors 556, 558 may be sewn into the fabric of the garment shell 500. The garment shell 500 may include a first electrical connector 556 (e.g., an adapter) configured to connect the battery 545 and/or controller to a second electrical connector 558 (e.g., a cable, wire, conductive thread, etc.).

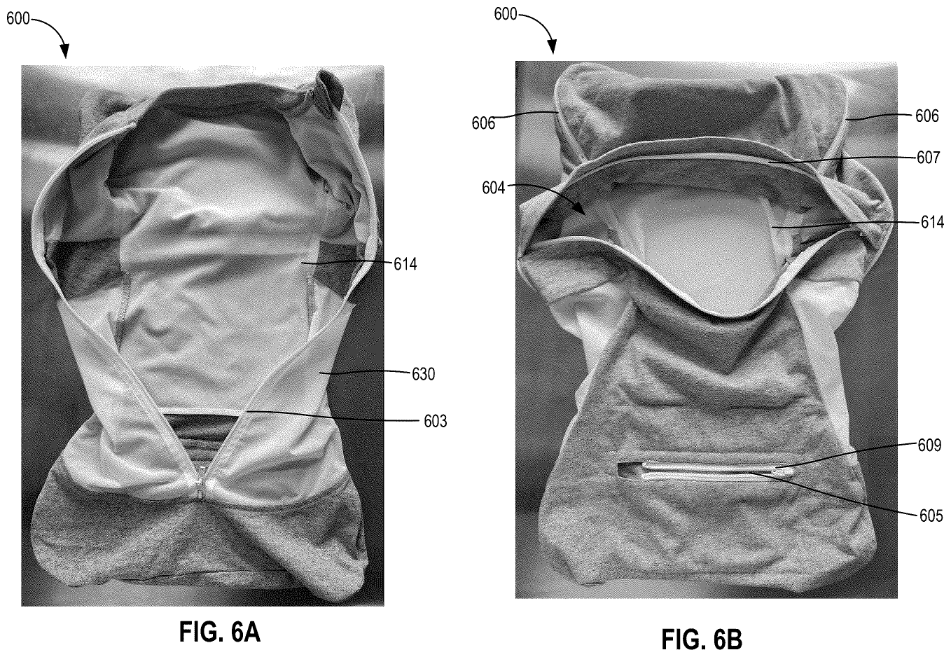

FIG. 5C is a photograph of the garment shell 500 showing the first pocket 504 in which the light panel is configured to be removably disposed, according to embodiments. As shown, the first pocket 504 includes a portion of fabric 514 (e.g., transparent fabric) sewn to an inner surface of a main body of the garment shell 500 such that the portion of fabric and the inner surface of the main body define a space therebetween. In some embodiments, the main body of the garment shell 500 and a first side of the portion of fabric 514 may define a first space in which the infant is disposed and the main body of the garment shell 500 and a second side of the portion of fabric 512 opposite the first side may define a second space separate from the first space in which the light panel is disposed. The first pocket 504 can include the fastener 507 to open and/or close the first pocket. As shown, the fastener 507 can be accessible to the user from inside the garment shell 500. The FIGS. 6A-6B show a front side and a back side, respectively, of a garment shell 600 configured to hold a light panel for delivery phototherapy to an infant, according to embodiments. As shown in FIG. 6A, the garment shell 600 includes a first fastener 503 configured to open and/or close the garment shell 600 such that the infant can be disposed inside. The first fastener 603 may open a main body of the garment shell 600, which defines a first space in which the infant can be disposed. The garment shell 600 further includes a breathable section 630 forming a portion of a front side of the main body of the garment shell 600. As shown in FIG. 6B, the garment shell 600 further includes a first pocket 604 defining a second space physically separate from the first space. In some embodiments, the light panel is configured to be removably disposed in the second space defined by the first pocket 604. The first pocket 604 may include a portion formed from a transparent material such that light can shine through the transparent material into the first space in which the infant is disposed. The garment shell 600 further includes a second pocket 605 defining a third space configured to receive one or more electrical components. For example, the second pocket 605 may be configured to receive at least one of a power source and/or a controller. The second pocket 605 includes a second fastener 609 configured to open and/or close the second pocket. As shown, the second pocket 605 can be disposed on a lower back side of the garment shell 500. The garment shell 600 may further include a pair of arm holes 606 including an adjustable portion or fastener to change a size of the arm holes 606 (e.g., a zipper). The garment shell 600 may be structurally and/or functionally similar to the garment shells 100, 300, 400, 500 and therefore certain details of the garment shell 600 are not described herein.

FIG. 7 shows a schematic diagram of a cross-sectional side view of a light panel 710 including a plurality of light sources 712 and a protective shell 720, according to embodiments. As shown, the light sources 712 (LED's) may be coupled to and/or disposed on a circuit board 716. The light sources 712 may include a distance D between adjacent light sources. In some embodiment, the protective shell 720 may have a thickness T. In some embodiments, the protective shell 720 may include one or more cut outs 722 disposed between the light sources 712. In some embodiments, the cut outs 722 may be in plane with the light sources 712. Alternatively, or additionally, the cut outs 722 can be in a different plane or extend into a different plane from the light sources 712. For example, the cut outs 722 can extend lower than the light sources, but remain outside of a light cone of the light sources 712. The cut outs 722 may be positioned such that the cut outs 722 are not disposed within a light cone of the light sources 712. As shown, the cut outs 722 may be semi-ovals (or semi-ellipsoids), however, it should be appreciated the cut outs 722 can have any suitable shape. In some embodiments, the circuit board 716 may transect the cut outs 722. The light sources 712 may each include a viewing angle a. In some embodiments, the distance D and the viewing angle a may be related by the equation:

$$tan ( \frac{a}{2} ) = \frac{D}{T} .$$

The distance D and the viewing angle a may be configured such that a total surface area at the edge of the protective shell 720 is illuminated (e.g., within the light cone). In some embodiments, the distance D and the viewing angle a may be configured such that greater than 80%, greater than 90%, greater than 95% of the total surface area at the edge of the protective shell 720 is illuminated. In some embodiments, the distance D may be about 1.5 cm to about 5 cm, inclusive of all ranges and subranges therebetween. In some embodiments, the viewing angle a may be about 100 degrees to 150 degrees, inclusive of all ranges and subranges therebetween. In some embodiments, the thickness T of the protective shell 720 may be in a range of about 0.5 mm to about 15 mm, inclusive of all ranges and subranges therebetween. The light source 712 shown in the dashed line may be optional because a total surface at the edge of the protective shell is illuminated by the other light sources 712 in the light panel 720. In some embodiments, the distance D, the thickness T, and the viewing angle a can be adjusted to evenly distribute the light emitted from the light panel. For example, these parameters may be adjusted such that an intensity of light is substantially evenly distributed (e.g., within 1%) of a desired value across the edge of the protective shell 720. The light panel 710 may be structurally and/or functionally similar to the light panels 110, 210, and therefore certain details of the light panel 710 are not described herein in FIG. 7.

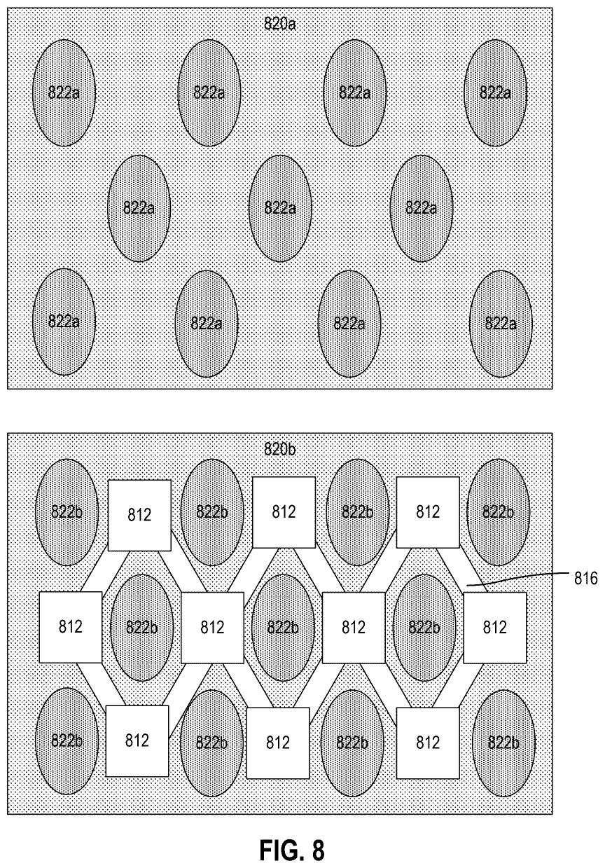

FIG. 8 is an exploded view of a light panel 810 including a plurality of light sources 812 and a protective shell 820a, 820b, according to embodiments. As shown, the light panel 810 includes a plurality of light sources 812 arranged in an ordered configuration (e.g., a grid configuration). The light sources 812 include one or more traces and/or a circuit board 816 connecting them. In some embodiments, the protective shell may include a first layer 820a defining a first set of indentations 822a (e.g., oval indentations). The light sources 812 and the circuit board 816 may be disposed on a second layer 820b of the protective shell. The second layer 820b may define a second set of indentations 822b between each of the light sources 812. A position and/or a shape of the first set of indentations 822a may correspond to a position and/or shape of the second set of indentations 822b. For example, the first set of indentations 822a may align with the second set of indentations 822b such that the first and second set of indentations form a sphere and/or an ellipsoid defining an inner volume containing air. In some embodiments, the indentations may function as a convergent lens to converge light emitted by the light sources 812, thereby increasing an irradiance of the light. While FIG. 8 shows the protective shell as having a first layer 820a and a second layer 820b each including indentations 822a, 822b, it should be appreciated that the protective shell may only include one protective layer and/or only one protective layer may include indentations. The light panel 810 may be structurally and/or functionally similar to the light panels 110, 210, 710 and therefore certain details of the light panel 810 are not described herein in FIG. 8.

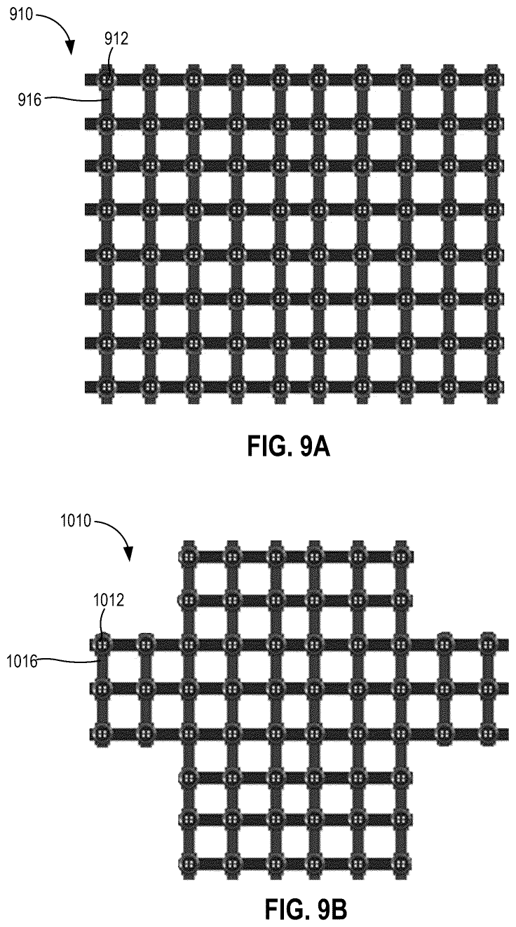

FIGS. 9A-9B show different configurations of light panels 910, 1010, according to embodiments. As shown in FIG. 9A, the light panel 910 forms a grid defining a plurality of square openings. At least some of the intersection points of the grid may include a light source 912. Each of the light sources 912 may be electrically connected and/or physically connected by the connectors (e.g., bands) 916. For example, each band 916 may include a conductive trace, a wire, a cable, etc. configured to electrically connected adjacent light sources 912. As shown, the light panel 910 forms a rectangle shape, although it should be appreciated the light panel 910 can form any suitable shape.

As shown in FIG. 9B, the light panel 1010 forms a grid defining a plurality of square openings. At least some of the intersection points of the grid may include a light source 1012. Each of the light sources 1012 may be electrically connected and/or physically connected by the bands 1016. For example, each band 1016 may include a conductive trace, a wire, a cable, etc. configured to electrically connected adjacent light sources 1012. As shown, the light panel 1010 forms a rectangle shape including a pair of projections. In some embodiments, the projections, extensions, or flaps of the light panel 1010 may be configured to wrap around from a back side of the infant towards a front side of the infant, e.g., when the light panel 1010 is disposed in a garment for delivery light therapy. The light panels 910, 1010 may be structurally and/or functionally similar to the light panels 110, 210, 710, 810, and therefore certain details of the light panels 910, 1010 are not described herein in FIGS. 9A-9B.

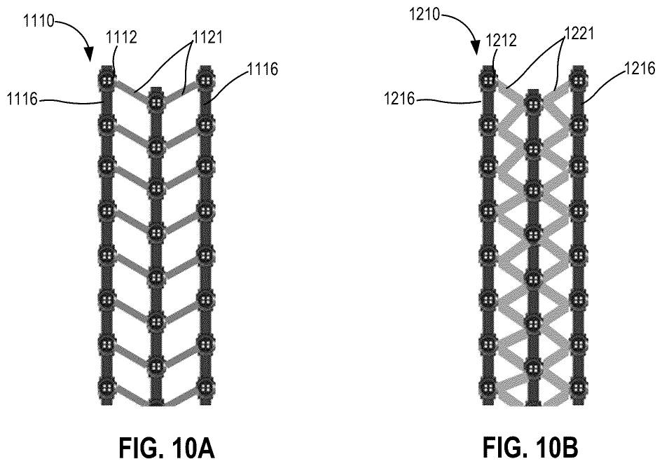

FIGS. 10A-10B show different configurations of light panels 1110, 1210, according to embodiments. As shown, the light panel 1110 forms a staggered grid configuration in which the light sources 1110 may be staggered. As shown, a first set of connectors 1121 may be physical connections and a second set of connectors 1116 may include electrical connections and/or include circuitry. For example, the first set of connectors 1121 may include silicone, and the second set of connectors 1016 may include circuits covered in silicone. The second set of connectors 1116 may provide data and/or power to and from the light sources 1112. In some embodiments, the second set of connectors 1016 may be configured to extend along a first direction (e.g., parallel to one another) and the first set of connectors 1121 may extend along a second and a third direction. For example, the first set of connectors 1121 may be arranged such that the light panel 1110 define a plurality of chevron-like shapes.

As shown in FIG. 10B, the light panel 1210 forms a staggered grid like configuration in which the light sources 1212 may be staggered. The light panel includes a first set of connectors 1221 that are physical connectors and a second set of connectors 1216 that include electrical connections and/or circuitry. In some embodiments, the first set of connectors 1221 may zig-zag. The zig-zag formation may provide stronger physical connections and can support a heavier weight of the light panel 1210. The zig-zag pattern may additionally increase irradiance of the light sources because the zig-zagging may increase overlap in light emitted between light sources. For LED design they want to make the light distribution more even. The light panels 1110, 1210 may both define openings (in other words they are open work panels). The light panels 110 may be structurally and/or functionally similar to the light panels 110, 210, 710, 810, 910, 1010, and therefore certain details of the light panel 1210, 1210 are not described herein in FIGS. 10A-10B.

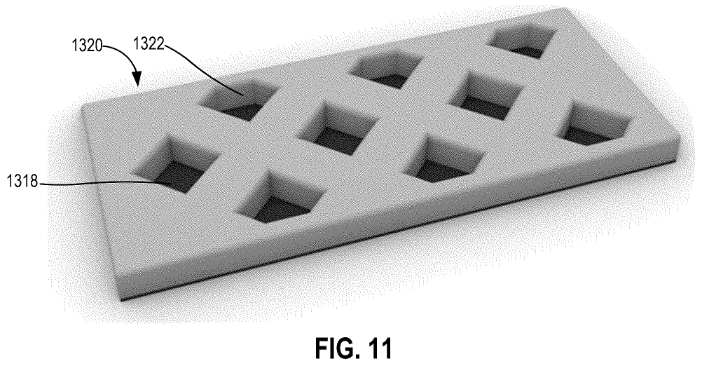

FIG. 11 shows a protective shell 1320 configured to be coupled to a light panel, the protective shell 1320 including a light absorber 1318, according to embodiments. As shown, the protective shell 1320 defines a plurality of cut outs 1322. The cut outs 1322 may extend from a first side of the protective shell 1320 through a second side of the protective shell 1320. As shown, the protective shell 1320 be openwork (e.g., form a lattice). The light absorber 1318 may not define openings. As shown, the light absorber 1318 is a solid piece of material. The light absorber may include an opaque or dark material such that light can be absorbed. In some embodiments, the light sources may be disposed along the edges of the cut outs 1322. In some embodiments, the light sources may be disposed at the intersection points of the openwork. In some embodiments, the light sources may be disposed along the edges of the openwork.

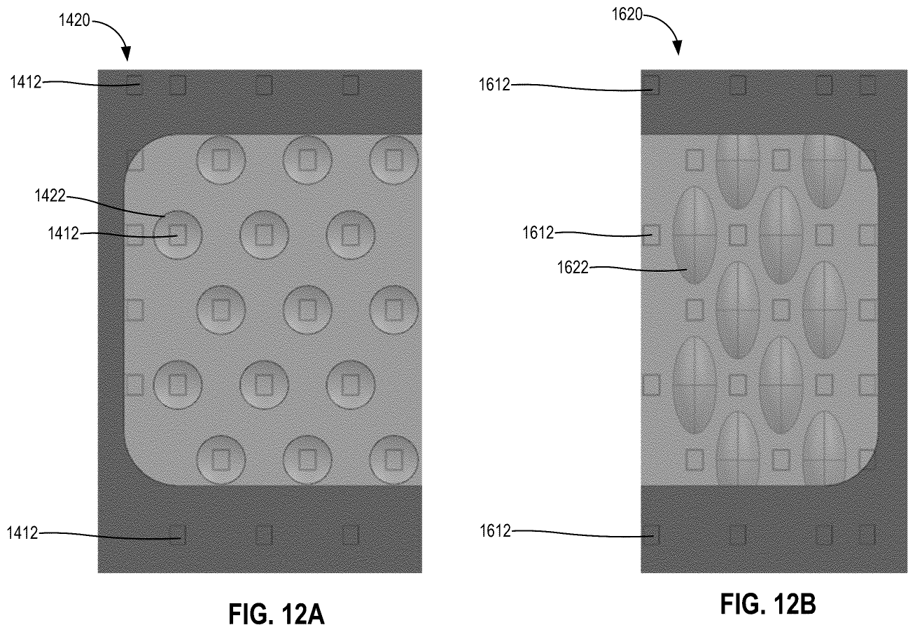

FIG. 12A is a diagram of a protective shell 1420 that includes cut outs 1422 disposed over the light sources 1412. As shown, the cut outs 1422 include a cross-sectional shape that is a circle. These cut outs 1422 may be disposed on an outer surface of the protective shell 1420 facing the skin of the infant and convex towards the skin of the infant (e.g., protrude towards the infant's skin). Therefore, this design may be uncomfortable to the infant because of the protrusion towards the chest of the infant. In contrast, FIG. 12B is a diagram of a protective shell 1620 that includes cut outs 1622 disposed between adjacent light sources 1612 and on an inner surface of the protective shell 1620, according to embodiments. As shown, the light sources 1412 can be arranged in an ordered pattern, and the cut outs 1622 may disposed therebetween. As shown, the cut outs 1622 may be oval (or have an oval cross-section). The cut outs 1622 may be contained within the protective shell 1620 such that a surface of the protective shell facing the chest of the infant is flat and/or smooth. This design may be more comfortable for the infant, as there are no protrusions pressing on the infant's chest. The protective shells 1420, 1620 may be structurally and/or functionally similar to any of the protective shells described herein, and therefore are not described in further detail in FIGS. 12A-12B.

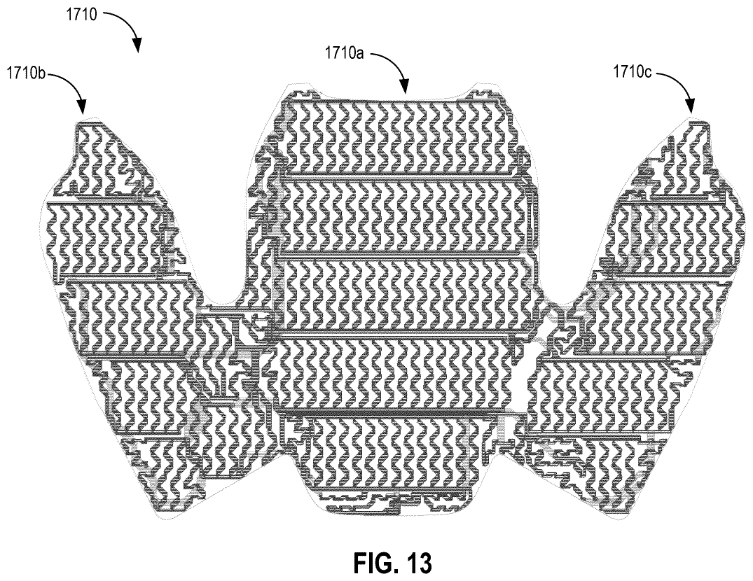

FIG. 13 is a diagram of a light panel 1700 having a shape that corresponds to a pocket of a garment shell, according to embodiments. The light panel 1710 may include a plurality of light sources and a plurality of connections 1716 connecting the light sources (e.g., physically and/or electrically). As shown, the connections 1716 may be zig-zag connections to increase a strength of the physical connection and/or support a heavier weight and/or to increase irradiance of the light panel 1710. The light panel 1710 may include a central section 1710a with a first extension (e.g., flap, projection, etc.) 1710b and a second extension 1710c extending laterally therefrom. The central section 1710a may be configured to align with a back of the patient, and the extensions 1710b, 1710c may each be configured to wrap around the patient and cover a portion of the chest of the patient.

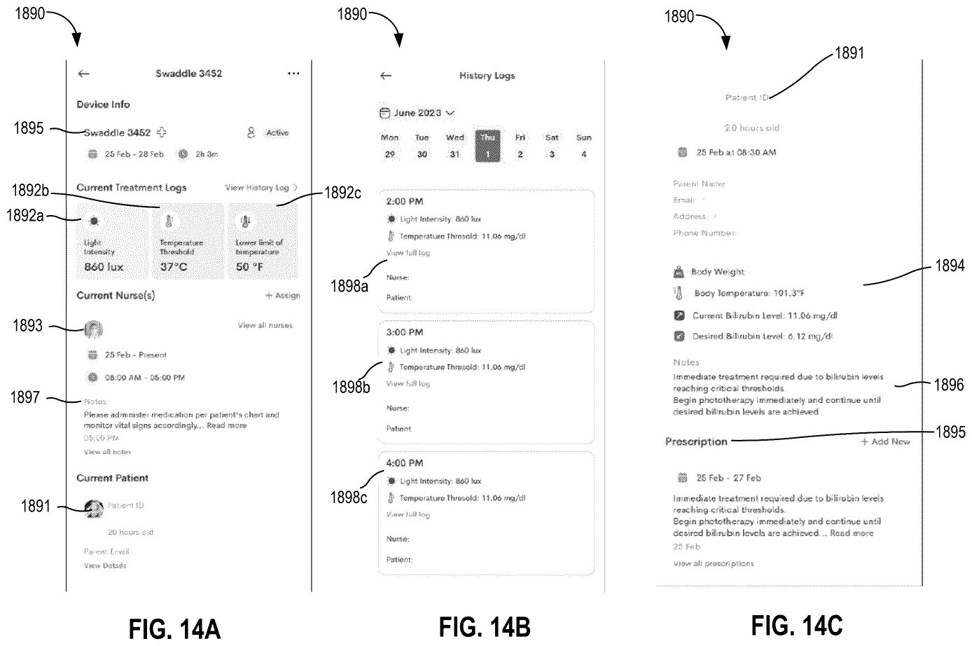

FIGS. 14A-14B show examples of a user interface 1890 (e.g., executable on an external device) for controlling a light panel of a phototherapy system, according to embodiments. As shown in FIG. 14A, the user interface 1890 may present information related to: (1) the patient identification (ID) 1891, the practitioner ID 1893 of the practitioner associated with the patient, the garment ID 1895 of the garment associated with the patient, treatment parameters 1892a, 1892b, 1892c, including sensor data from sessions, and/or notes 1897 from sessions. For example, the user interface 1890 can show the light intensity 1892a delivered during a selected session, an upper temperature threshold 1892b used during the selected session, and/or a lower temperature threshold 1892c used during the selected session. The user interface 1890 may be configured such that the user can select icons for more information. In some embodiments, the user interface 1890 may be configured to receive user inputs. User inputs can include selections of parameters for the light therapy session (e.g., duration, intensity), notes related to a session, and/or inputs to stop or start power to the light panel.

As shown in FIG. 14B, the user interface 1890 may allow the user to select a date, and the user interface 1890 may display a session log including a plurality of session summaries 1898a, 1898b, 1898c. The session summaries 1898a-1898c may include information related to the light therapy session(s) delivered on the date. As shown in FIG. 14C, the user interface may be configured to display a profile page for the patient including, for example, the patient ID 1891, information related to the patient 1894, notes related to the patient 1896, and/or a prescription 1895 associated with the patient for light therapy sessions. The user interface may provide an easy way for the caretaker of the infant to manage the phototherapy treatment using the phototherapy system. The user interface can allow real time or near real-time updates to be displayed to the user, thereby enabling the user to safely monitor the delivery of phototherapy sessions.

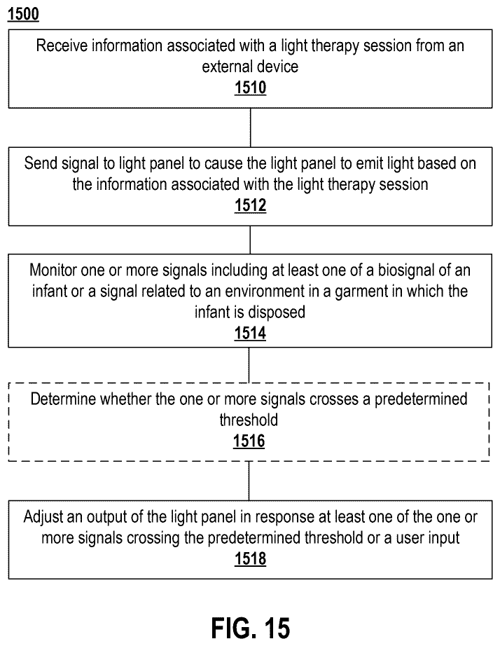

FIG. 15 is a flow chart diagram of method 1500 for delivery phototherapy using a phototherapy system, according to embodiments. Although method 1500 is described herein with respect to phototherapy system 1000, it should be appreciated that method 1500 is applicable for any phototherapy system described herein. The method 1500 may include receiving information associated with a light therapy session from an external device 180, at 1510. The information may include at least one of a duration of the session, a predetermined light intensity to be administered, a bilirubin level of the patient, a session history of the patient, etc. In some embodiments, a controller and/or an external device 180 coupled to the controller 150 may be configured to receive the information associated with the light therapy system 1000. In some embodiments, the information associated with the light therapy session may be a user input. At 1512, the method includes sending a signal to a light panel 110 (e.g., an LED panel) to cause the light panel 110 to emit light based on the information associated with the light therapy session. For example, the controller 150 may send a signal (e.g., voltage and/or current signal) such that the light panel 110 emits may light at the predetermined light intensity and/or for the predetermined amount of time. In some embodiments, a user can adjust parameters of the light therapy session (e.g., via the user interface). At 1514, the method includes monitoring one or more signals including at least one of a biosignal of an infant 102 or a signal related to an environment of a garment 100 in which the infant 102 is disposed. For example, the controller 150 may be configured to monitor (e.g., via the sensors 140) a temperature inside the garment 100 and/or a bilirubin level of the infant 102. The method may optionally include determining the one or more signals crosses a predetermined threshold, at 1516 (e.g., to keep the temperature below a predetermined threshold and/or the light intensity within a predetermined range). In some embodiments, the controller 150 may additionally or alternatively be configured to receive, before or during the light therapy session, information associated with a user input (e.g., from the external device) and/or the controller 150 may receive the user input directly.

The method 1518, may include adjusting an output of the light panel 110 in response to at least one of the one or more signals crossing the predetermined threshold or a user input. In some embodiments, if the at least one of the one or more signals crosses the predetermined threshold, the method may include increasing and/or decreasing an intensity of light or turning off the light panel 110. For example, if the temperature increases above the predetermined threshold (e.g., 40 degrees C.), the method may include reducing the light intensity emitted by the light panel 110 and/or turning off the light panel 110. In some embodiments, the controller 150 may automatically adjust the output. In some embodiments, the user may manually adjust the output. In some embodiments, the method 1500 may optionally include alerting the user (e.g., via the external device 180) that a signal crossed a respective predetermined threshold. The method 1500 may further include storing information related to the light therapy session in a memory (e.g., on the controller 150 and/or on the external device 180.) In some embodiments, the information related to the light therapy session stored may inform future session. The method 1500 described herein, allows a user to remotely monitor and/or easily administer light therapy to an infant and to receive real-time and/or near real-time updates associated with the light therapy.

Various concepts may be embodied as one or more methods, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments. Put differently, it is to be understood that such features may not necessarily be limited to a particular order of execution, but rather, any number of threads, processes, services, servers, and/or the like that may execute serially, asynchronously, concurrently, in parallel, simultaneously, synchronously, and/or the like in a manner consistent with the disclosure. As such, some of these features may be mutually contradictory, in that they cannot be simultaneously present in a single embodiment. Similarly, some features are applicable to one aspect of the innovations, and inapplicable to others.

In addition, the disclosure may include other innovations not presently described. Applicant reserves all rights in such innovations, including the right to embodiment such innovations, file additional applications, continuations, continuations-in-part, divisionals, and/or the like thereof. As such, it should be understood that advantages, embodiments, examples, functional, features, logical, operational, organizational, structural, topological, and/or other aspects of the disclosure are not to be considered limitations on the disclosure as defined by the embodiments or limitations on equivalents to the embodiments. Depending on the particular desires and/or characteristics of an individual and/or enterprise user, database configuration and/or relational model, data type, data transmission and/or network framework, syntax structure, and/or the like, various embodiments of the technology disclosed herein may be implemented in a manner that enables a great deal of flexibility and customization as described herein.

All definitions, as defined and used herein, should be understood to control over dictionary definitions, definitions in documents incorporated by reference, and/or ordinary meanings of the defined terms.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the embodiments, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

While specific embodiments of the present disclosure have been outlined above, many alternatives, modifications, and variations will be apparent to those skilled in the art. Accordingly, the embodiments set forth herein are intended to be illustrative, not limiting. Various changes may be made without departing from the spirit and scope of the disclosure. Where methods and steps described above indicate certain events occurring in a certain order, those of ordinary skill in the art having the benefit of this disclosure would recognize that the ordering of certain steps may be modified and such modification are in accordance with the variations of the invention. Additionally, certain of the steps may be performed concurrently in a parallel process when possible, as well as performed sequentially as described above. The embodiments have been particularly shown and described, but it will be understood that various changes in form and details may be made.

1. An apparatus for delivering light therapy to an infant, the apparatus comprising:
a garment shell configured to be opened such that the infant can be disposed inside the garment shell, the garment shell including:
a first pocket disposed on an inner surface of the garment shell, the first pocket defining a space in which a light panel is configured to be removably disposed, the light panel including a plurality of light sources configured to emit light at a predetermined wavelength and intensity,
the space of the first pocket extending from a back side of the garment shell to a front side of the garment shell such that when the infant is disposed inside the garment shell and the light panel is disposed in the first pocket, a first portion of the light panel is aligned with a back of the infant and a second portion of the light panel is aligned with a chest of the infant;
a second pocket defining a space on the garment shell, the second pocket configured to hold a battery and a controller operatively coupled to the light panel; and
a mesh forming a portion of the garment shell and configured to allow at least one of light or heat emitted by the light panel to exit the garment shell through the mesh.

2. The apparatus of claim 1, wherein the garment shell further includes:
a first zipper extending along the front side of the garment shell, the first zipper configured to be actuated to open the garment shell such that the infant can be disposed inside the garment shell.

3. The apparatus of claim 1, wherein the garment shell further includes:
a movable flap attached to a side of the garment shell, the movable flap having a first configuration in which the movable flap does not cover the mesh and a second configuration in which the movable flap covers the mesh to block light emitted by the light panel from exiting the mesh.

4. The apparatus of claim 3, wherein the movable flap is movable between the first configuration and the second configuration by a user holding the infant.

5. The apparatus of claim 1, wherein the mesh at least partially aligns with a stomach of the infant when the infant is disposed in the garment shell.

6. The apparatus of claim 1, wherein the light panel includes one or more sensors coupled thereto, the one or more sensors configured to monitor at least one of a biosignal of the infant or an environment inside the garment shell.

7. The apparatus of claim 6, wherein the biosignal of the infant may include a bilirubin level of the infant.

8. The apparatus of claim 5, wherein the environment inside the garment shell includes at least one of a location of the garment shell, a temperature inside the garment shell, a temperature of the light panel, or a humidity inside the garment shell.

9. The apparatus of claim 1, wherein the garment shell includes double-layered cotton.

10. The apparatus of claim 1, wherein a portion of the first pocket includes a transparent material such that light emitted from the light panel reaches skin of the infant when the infant is disposed in the garment shell.

11. The apparatus of claim 1, wherein the garment shell further includes:
a pair of arm holes configured to receive arms of the infant therethrough, a size of each of the pair of arm holes being adjustable such that outer edges of each of the pair of arm holes abut the arms of the infant to prevent light from escaping the inside of the garment shell through the pair of arm holes; and
a neck hole configured to receive a head of the infant therethrough, wherein the neck hole includes a light shielding collar to prevent light from escaping the inside of the garment shell through the neck hole.

12. The apparatus of claim 1, wherein the garment shell is a swaddle.

13. The apparatus of claim 1, wherein the plurality of light sources includes light emitting diodes (LEDs).

14. The apparatus of claim 1, wherein the garment shell includes an active heat dissipation mechanism including a fan.

15. An apparatus, comprising:
a flexible circuit board;
a plurality of light sources arranged in an ordered configuration, the plurality of light sources coupled to a first side of the flexible circuit board; and
a flexible protective shell coupled to the flexible circuit board and the plurality of light sources, the protective shell disposed on a second side of each of the light sources of the plurality of light sources and the second side of the flexible circuit board, the flexible protective shell including a plurality of cut outs disposed between at least some adjacent light sources of the plurality of light sources,
the plurality of light sources configured to be disposed near a surface of skin of an infant and emit light on the surface of the skin of the infant, the plurality of light sources having a viewing angle and being spaced from one another such that an intensity of light emitted from the plurality of light sources is above a predetermined threshold at the surface of the skin of the infant.

16. The apparatus of claim 15, wherein a viewing angle of each light sources of the plurality of light sources is in a range between 0 degrees and 180 degrees.

17. The apparatus of claim 15, wherein the protective shell includes at least one of silicone or polyvinyl chloride (PVC).

18. The apparatus of claim 15, wherein the protective shell includes a first layer and a second layer joined together, the first layer disposed on the second side of the light sources and the second layer disposed on the second side of the flexible circuit board, the first layer defining a first portion of the plurality of cut outs and the second layer defining a second portion of the plurality of cut outs.

19. The apparatus of claim 15, wherein at least a portion of the protective shell covering the second side of the plurality of light sources is transparent.

20. The apparatus of claim 15, wherein the protective shell further includes a light-reflective layer, the light-reflective layer disposed on the second side of the flexible circuit board such that stray light from the plurality of light sources is reflected back to the surface of the skin of the infant.
